(19) United States
(12) Patent Application Publication
(10) Pub. No.: US 2015/0226722 A1
(43) Pub. Date: Aug. 13, 2015

(54) Optical Trace Chemical Detection for Analysis of Latent Prints

(71) Applicant: BAE Systems Information and Electronic Systems Integration Inc., Nashua, NH (US)

(72) Inventors: Somnath Sengupta, Ellicott City, MD (US); Christopher S. Badorrek, Hagerstown, MD (US); Michael R. Beversluis, Tucson, AZ (US); Igor I. Smolyaninov, Columbia, MD (US); Laura A. Swafford, Baltimore, MD (US)

(73) Assignee: BAE Systems Information and Electronic Systems Integration Inc., Nashua, NH (US)

(21) Appl. No.: 13/951,519

(22) Filed: Jul. 26, 2013

Related U.S. Application Data
(60) Provisional application No. 61/796,544, filed on Nov. 13, 2012.

(51) Int. Cl. G01N 33/28; G01N 21/31

(52) U.S. Cl. CPC G01N 33/2835; G01N 21/31; G01N 2201/06113

(57) ABSTRACT

A method for optical analysis of otherwise invisible latent prints on a wide variety of surfaces and trace chemicals contained within the oils of a print. The invention provides the capability to monitor absorption/reflection infrared bands that are unique to print oils and chemicals of interest that have long or short wave infrared signatures, such as explosives, inorganic oxidizers, drugs, environmental markers, and biomarkers.

### RELATED APPLICATIONS

This application claims the benefit of U.S. Provisional Applications No. 61/796,544, filed Nov. 13, 2012 which is herein incorporated by reference in its entirety for all purposes.

### FIELD OF THE INVENTION

This present invention relates to forensic science and more particularly to the optical analysis of latent prints and detection of trace chemicals.

### BACKGROUND OF THE INVENTION

Current latent print analysis allows investigators to identify persons of interest by comparing the ridge detail patterns of prints with databases of known prints. However, frequently the detected print does not match any print on file. When this occurs, the print does not assist in investigation until such time as the print can be linked to an individual through outside means (i.e., if the suspect gives a print as part of the current or other investigation). Moreover, in sensitive site exploitation (SSE), when print powder cannot be used, investigators are limited to imaging prints in the visible range, which makes most prints inaccessible in these situations.

A need, therefore, for an improved method of print analysis.

### SUMMARY OF INVENTION

One general embodiment of the invention is an optical method for both imaging otherwise invisible prints on a wide variety of surface and identifying trace chemicals contained within the oils of a print.

The skin found on the fingers, palms and soles of the feet of humans is known as friction skin. This skin is unique because it does not have hair follicles or oil glands, and because it is composed of ridges that arc believed to be adapted for increased friction to help when handling various objects and walking. These so-called friction ridges are composed of rows of sweat pores that constantly secrete perspiration. This perspiration—along with grease and oil transferred from other parts of the body—adheres to the friction skin and is transferred from the skin to other surfaces when contact is made with objects. The transferred outline of the friction ridges is what is known as a latent print. In addition to leaving latent prints, body oils and perspiration also adsorb trace quantities of loose material that an individual comes in contact with. Usually this is organic material (including nicotine from smoke, drugs, and many explosives) or powders (including drugs and organic and inorganic explosives). These trace chemicals are then deposited as part of the latent print. If detectable, they yield insight into the suspect's activities.

These chemicals can be: (a) biological indicators that relay information about the suspect's gender, age, race, medical status (e.g., diabetic), etc; (b) chemical indicators that relay information on habits or where the suspect has been (e.g., suspect is a smoker or has been near a specific manufacturing plant); and (c) chemical indicators that relay information on illegal activities (e.g., suspect has handled illicit drugs or materials found in improvised explosive devices (IEDs)).

By identifying these trace chemicals, insight can be gained into a suspect's identity and activities even when the print cannot be identified by ridge detail. This will lead to more insight into the individual's chemical history that would provide additional information for sensitive site exploitation, including evidence of illicit activity. Because the method is optical, it can be used in situations in which the investigator does not wish his or her presence known.

One general aspect of the invention is a method of print detection and trace chemical identification using spectral imaging of the unaltered latent print. Every chemical has a distinct spectral signature. The spectrum could be measured in the ultraviolet (UV), visible (VIS), or near-infrared (NIR) to detect electronic transitions, or short wave, mid wave and long wave infrared (SWIR, MWIR, and LWIR) to detect molecular vibrations.

in one embodiment, multispectral or hyperspectral imaging of prints would allow the investigator to distinguish the print from the background material, as the print and background would have different spectra. In addition, the spectrum of the print would be the superposition of the spectra of all the chemicals contained within the print. By applying a chemical detection algorithm, this spectrum can be deconvoluted into the constituent chemicals. This technique is advantageous in that it could be used in situations in which print powder is not desired due to logistical considerations or when covert print identification is desired.

There are four major advantages to this technique. First, the chemicals can be used to narrow down the identity of the suspect based on biological markers, even if the suspect's prints are not available for comparison to the unidentified print Second, the chemicals may provide into the suspect's habits and/or where the suspect may have been before leaving the print. Third, the chemicals may provide evidence of illicit activity. Fourth, the above-mentioned process does not leave evidence of SSE.

Although the application here is trace chemical analysis of latent prints, a spectrometer capable of performing this analysis would also be able to identify trace chemicals on surfaces in general from other body fluids. If one can detect trace chemicals related to bio markers, than this could potentially be used to diagnose disease. For example, ketones could indicate diabetes. Similar biomarkers are being used to diagnose disease in breath analysis; this would be another way to get at the same answer.

### BRIEF DESCRIPTION OF THE DRAWINGS

The present invention is further described with reference to the accompanying drawings wherein:

FIG. 1 is a graph showing DKR spectra of prints from two people on gold substrates.

FIGS. 2A-2D show identification of prints on different surfaces at different LWIR wavelengths.

FIGS. 3A and 3B show the detection of trace potassium chlorate in the presence of silicon dioxide.

FIG. 3C shows a graphical representation of potassium chlorate detection in the presence of noise.

FIG. 4A shows the detection of ammonium nitrate in a print using differential SWIR imaging at 1.55 and 1.4 um.

FIG. 4B shows trace amounts of urea and potassium chlorate in the presence of silicon dioxide using a LWIR microbolometer camera.

FIG. 4C shows a graph of the detection of urea.

FIG. 4D shows a graph of the detection of potassium chlorate.

FIG. 5 is a showing an example architecture for processing.

### DETAILED DESCRIPTION OF THE PREFERRED EMBODIMENT

One general aspect of the invention is the detection of specific molecular vibrations within chemicals, as all organic molecules (and many inorganics) have unique vibrational energy reflection signatures in SWIR-LWIR. Thus, the invention provides the capability to monitor absorption/reflection IR bands that are unique to print, oils and chemicals of interest that have LWIR signatures, such as military or homemade explosives (HMEs) inorganic oxidizers. To our advantage, there is minimal overlap between the IR bands in print oils versus those HMEs, precursors, or oxidizers. A substrate, being composed of a separate set of chemicals, will necessarily have a spectrum that is distinct from that of the print or trace chemicals. Therefore, by identifying spectral features of print oils and chemicals of interest, one embodiment of this invention can distinguish prints and trace chemicals from background materials.

The invention is further discussed in reference to the following figures and working examples:

### EXAMPLE 1

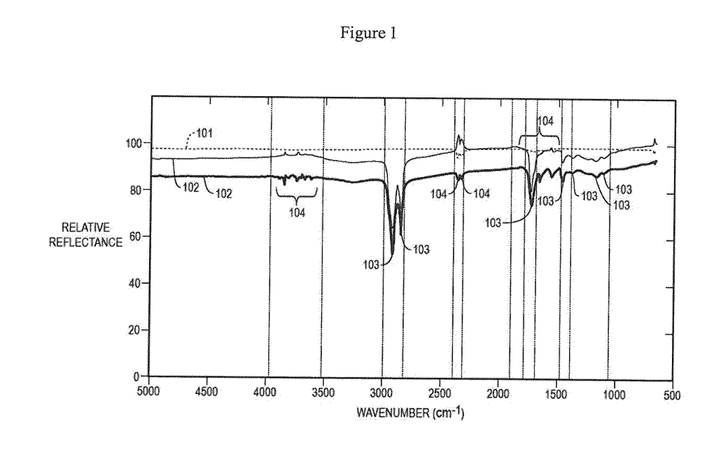

FIG. 1. is a graphical representation showing the distinction between the IR spectra of prints from two people and the god used as substrate. Shaded regions indicate atmospheric and print absorption bands. The y-axis shows the relative reflectance and the wavenumber is indicated along the x-axis.

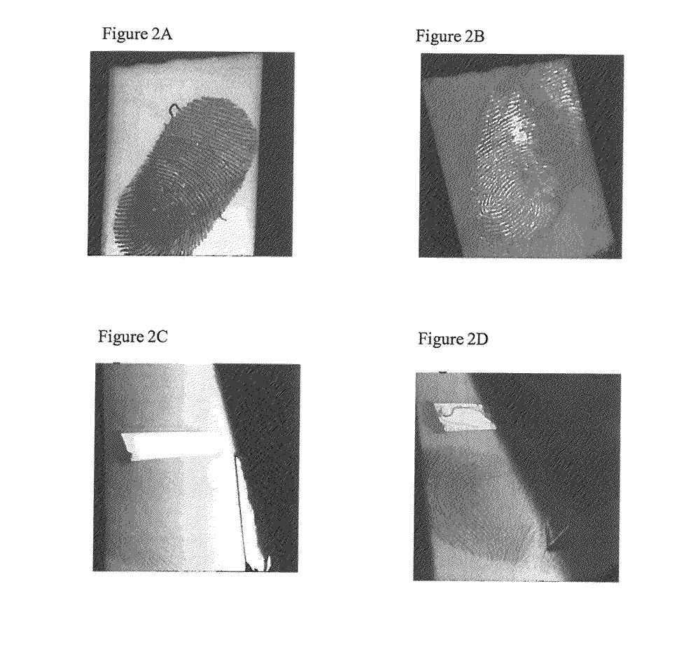

FIG. 2 then demonstrates that prints have different IR spectra than substrate materials. Since IR spectra arc complex, the most accurate method for distinguishing between print and substrate material is through hyperspectral imaging (HSI), in which a complete a spectrum (LWIR, MWIR SWIR, or any combination thereof) is obtained for each pixel in the image. However, HSI is a time-intensive process and is not practical for locating prints on a surface. To locate prints, imaging at a single wavelength filtered imaging) or a few wavelengths (multispectral imaging) is preferred.

### EXAMPLE 2

FIG. 2 provides identification of prints on different surfaces at different LWIR wavelengths. Because of the different chemical natures of the substrates, different wavelengths are necessary to obtain clear images of the prints. FIG. 2A is imaged at 9.64 um on a glass slide. FIG. 2B is imaged at 8.55 um, also on a glass slide. In this case, an interrogation wavelength of 9.64 um provides the superior image. FIG. 2c is imaged at 9.64 um on a plastic cup. FIG. 2D is imaged at 8.55 um on a plastic cup. In this case, an interrogation wavelength of 8.55 um provides the superior image.

FIG. 2 then demonstrates that either single-wavelength or multispectral LWIR imaging can be used to locate and image prints. In this embodiment, LWIR was selected due to the presence of multiple spectral features in the print oil spectrum; however, another embodiment will also include SWIR scanning capability for compounds active primarily in SWIR. In both embodiments since each surface has its own distinct LWIR/SWIR features, no single wavelength would be successful in locating and imaging prints on all surfaces. Therefore, multispectral imaging is preferred for print location, as the appropriate choice of a few wavelengths should yield contrast between print and substrate for most surfaces. It is anticipated that a single wavelength could be used to scan a single surface to locate prints. Generally though, multispectral imaging balances the need for short scan times with the desire for a high probability of detection.

Once prints are located and imaged for suspect identification, SWIR HSI and LWIR HSI are used to distinguish chemicals associated with each print. In one embodiment, SWIR HSI was used because many of the chemicals associated with homemade explosives (HMEs) have distinct features in this region. In fact, SWIR has already been utilized for differentiating chemicals of interest (i.e. explosive precursors, oxidizers, etc.) from other man-made materials and natural background. In this embodiment, LWIR HSI was then used to increase probability of detection P_{d} and decrease the probability of false alarms (Pfₐ) by providing a means to differentiate target chemicals from confusers in a complex environment.

### EXAMPLE 3

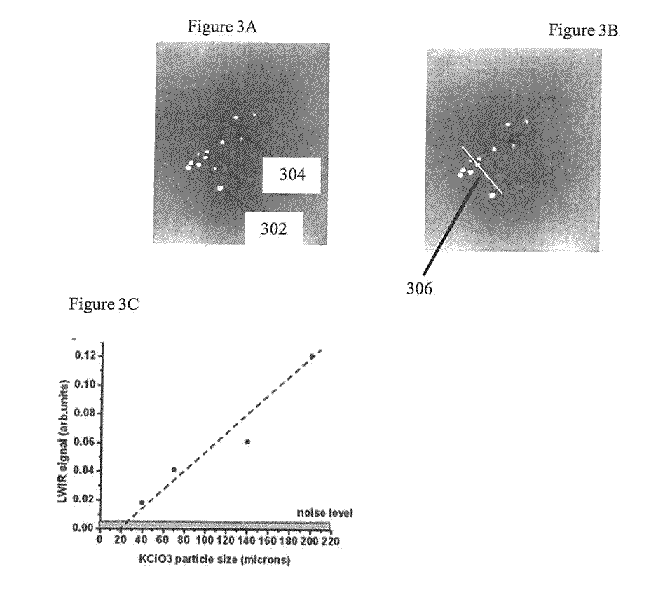

FIG. 3C is a graphical representation of this means of differentiating target chemicals. Specifically, FIG. 3A shows the detection of trace potassium chlorate 302 in the presence of silicon dioxide confusers 304 through the use of a carbon dioxide (CO₂) laser (10.6 um) and a LWIR microbolometer. Potassium chlorate and silicon dioxide have overlapping SWIR spectra, so SWIR HSI may not be able to distinguish an ingredient in explosives (potassium chlorate) from sand (silicon dioxide). LWIR imaging, however, shows a distinctly different spectrum. In these LWIR images, the potassium chlorate appears white and silicon dioxide appears dark.

One embodiment of the invention anticipates that targeting of non-SWIR-active chemicals is desired, and therefore in this embodiment LWIR imaging is used as the primary means of chemical identification, while SWIR imaging is used to distinguish between chemicals of interest and confusers. This methodology is anticipated to be more useful in identifying organic materials, including biomarkers and prescription or illicit drugs.

### EXAMPLE 4

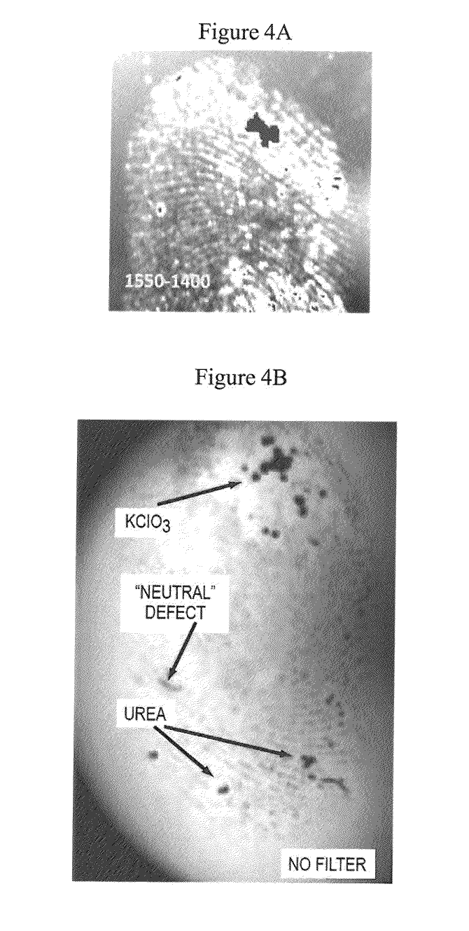

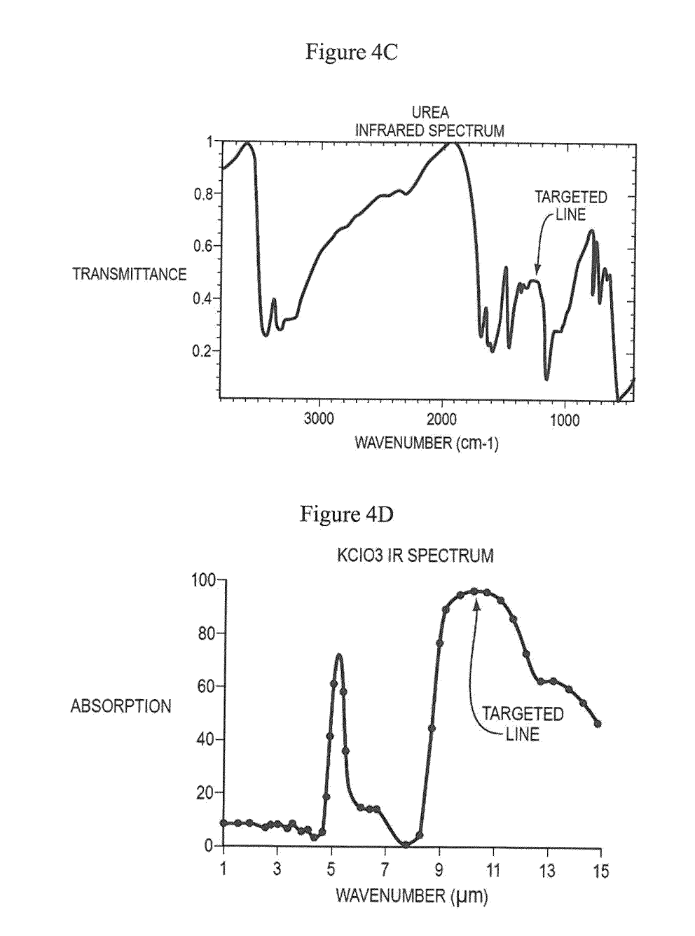

FIG. 4A-D shows the results of tests conducted to establish the non-contact interrogation capabilities of embodiments of the invention using SWIR/LWIR for certain chemicals of interest. In FIG. 4A the first latent print 12, trace amounts of ammonium nitrate 14 were detected using a SWIR camera with a broadband illumination source. In FIG. 4B trace amounts of urea 16 and potassium chlorate 18 in the presence of silicon dioxide 20 were detected using a LWIR microbolometer camera. The proof-of-principle demonstrations of non-contact print detection, imaging and chemical analysis were performed using a commercial, off-the-shelf (COTS) LWIR camera (FLIR), a COTS SWIR camera (SUI Goodrich), a broadband light source with narrow band pass filters, and a CO₂ laser tuned at 10.6 um.

In one embodiment initial tests of imaging parameters indicated that prints can be identified and imaged in both LWIR and SWIR over a range of distances (1 cm-1 m), and that 60-75° is an optimal illumination/viewing angle. furthermore data suggests that by using a tunable LWIR source, it is possible to focus on unique peaks of interest to distinguish chemicals like potassium chlorate from confusers like silicon dioxide (see earlier discussion of FIG. 3). In terms of sensitivity, it is possible to detect particles >20 um in diameter for potassium chlorate with the current FLIR LWIR microbolometer.

The following are examples of potential pieces of equipment which might be utilized in various embodiments of the invention.

LWIR camera—For three embodiments, uncooled microbolometer technology was chosen to attain low size, weight, and power needed for man-portable systems because cooled mercury cadmium telluride (MCT) detectors will not be able to attain the required SWaP. For the other embodiment, the camera chosen was selected for its applicability to man-portable systems. The SXGA format SMART Chip microbolometer offers the best resolution combined with low SWaP-C; however, the use of other cameras such as ANTLIR and VGA Smart Chip are also anticipated.

Quantum cascade lasers—Various working examples of this invention revealed that active lighting is necessary for LWIR imaging. For this reason, in one embodiment, active illumination is provided, as well as tenability, through Daylight Solutions' Uber Tuner lasers. QCLs have previously been used in combination with LWIR microbolometers for successful stand-off explosives detection (Bernacki, B. and Phillips, M., 2010, Proc. of SPIE, Vol. 7665). In one embodiment, two COTS lasers, UT-9 and UT-10, provide a tunable range of 8.3-12.4 um.

SWIR camera—For embodiments utilizing SWIR imaging, a SWIR HSI sensor was used. One embodiment utilizes a sensor with combined precision and rugged diamond-turned optical housing and imaging lens along with a COTS SWIR focal plane array (FPA).

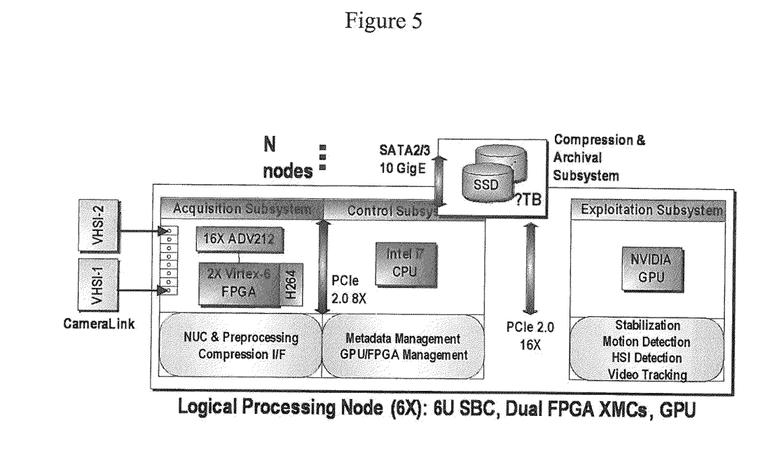

Hardware Architecture—FIG. 5 shows one possible embodiment of an on-board processor logical node which implements the video pipelines (FPGA), hosts the control software (CPU), and executes key hyperspectral detection algorithms (GPU). A similar heterogeneous mix of processing components (FPGA/CPU/GPU) was developed to be an appropriate, efficient (watts/FLOP), and effective architecture for processing high data volumes in real, or near real, time. The requirement for a high grade processor is necessitated by the desire for an accurate method of distinguishing prints from a variable background, identifying chemical threat compounds via advanced embedded algorithms, and delivering that information as quickly as possible.

### EXAMPLE 5

In one embodiment, multispectral imaging is utilized. The advantages to this imaging mode are: (1) rapid scan of surfaces to locate prints and (2) ability to target detection of specific chemicals of interest. However, multispectral imaging is less precise than hyperspectral imaging and cannot target a wide range of chemicals simultaneously.

For this reason, in another embodiment, hyperspectral imaging (HSI) can be utilized (as opposed to a multi-spectral approach). There are several advantages to this: (1) HSI enables advanced algorithms to detect known chemicals, (even when mixed and in low concentrations), via their spectral signatures; (2) HSI allows for the detection of newly identified materials of interest without changing any physical components (such as filters and illuminators in other systems); (3) most importantly, HSI allows for the reduction or illumination of false alarms in comparison to multispectral imaging by differentiating background materials and confusers from true threats due to the additional information afforded by covering a broader range with high spectral resolution; and (4) HSI has been proven to be effective at detecting materials of interest in numerous airborne and ground based systems.

The task of detecting chemicals within a latent print can be performed by what is essentially a search function, in which the spectral content of each pixel is compared to the known spectra of various chemicals in a library. This function is comprised of two elements: (i) lighting conditioning (reflectance) and (ii) spectral decomposition/identification.

There are several spectral libraries that consist of ˜8000 spectra that could be used for reference purposes. The chemicals represented are both natural and man-made, benign and dangerous. After calibrating the library for sensor response, scene illumination can be accounted for by using a scene-based statistical method rather than a first-principles calculation.

Spectral Decomposition/Identification: One embodiment integrates (1) adaptive coherence/cosine estimator (ACE), and (2) spectral decomposition methods which use known spectral signatures to identify chemicals present in a latent print and have significant complementary strengths for overcoming sources of false alarms. For example, ACE measures the probability that the spectrum of some material of interest is present in the pixel and requires no a priori knowledge other than the spectrum of the chemical of interest. Complementary, spectral decomposition essentially solves the inverse linear problem, and finds the linear combination of library entries that best accounts for the observed spectrum (‘best’ here being used in the sense of least squares).

While the present invention has been described in connection with the preferred embodiments of the various figures, it is to be understood that other similar embodiments may be used or modifications and additions may be made to the described embodiment for performing the same function of the present invention without deviating there from. Therefore, the present invention should not be limited to any single embodiment, but rather construed in breadth and scope in accordance with the recitation of the appended claims.

What is claimed is:

1. A method for optical analysis of latent prints comprising the steps of:
scanning a surface for a chemical;
identifying said chemical optically;
obtaining a spectrum of said chemical; and
comparing a spectral content of said chemical to a plurality of known spectral identities.

2. The method for optical analysis of claim 1 wherein the step of scanning a surface further comprises multispectral imaging.

3. The method for optical analysis of claim 2 further comprising the step of detecting electronic transitions.

4. The method for optical analysis of claim 2 further comprising the step of detecting molecular vibrations.

5. The method for optical analysis of claim 3 wherein the step of scanning a surface further comprises scanning for a ultraviolet spectrum of said chemical.

6. The method for optical analysis of claim 3 wherein the step of scanning a surface further comprises scanning for a visible spectrum of said chemical.

7. The method for optical analysis of claim 3 wherein the step of scanning a surface further comprises scanning for a near infrared spectrum of said chemical.

8. The method for optical analysis of claim 4 wherein the step of scanning a surface further comprises scanning for a short wave infrared spectrum of said chemical.

9. The method for optical analysis of claim 4 wherein the step of scanning a surface further comprises scanning for a midwave infrared spectrum of said chemical.

10. The method for optical analysis of claim 4 wherein the step of scanning a surface further comprises scanning for a long wave infrared spectrum of said chemical.

11. The method for optical analysis of claim 10 wherein the step of scanning for a long wave infrared spectrum further comprises providing active lighting with quantum cascade lasers.

12. The method for optical analysis of claim 1 further comprising the step of differentiating a target chemical from a confuser.

13. The method for optical analysis of claim 12 wherein the step of differentiating a target chemical from a confuser further comprises using a short wave infrared HSI to distinguish said target chemical from said confuser.

14. The method for optical analysis of claim 12 wherein the step of differentiating a target chemical from a confuser further comprises using a long wave infrared HSI to distinguish said target chemical from said confuser.

15. The method for optical analysis of claim 12 further comprising the step of utilizing an uncooled microbolometer.

16. The method for optical analysis of claim 1 further comprising the steps of:
distinguishing prints from a variable background,
identifying said chemical; and
delivering an identification for said chemical.

17. The method for optical analysis of claim 16 wherein the step of comparing the spectral content further comprises the steps of:
referring to a spectral library;
calibrating a spectral library for sense spouse;
accounting for scene illumination;
comparing a lighting conditioning of said trace chemical to said plurality of known spectral identities; and
comparing a spectral decomposition of said trace chemical to said plurality of known spectral identities.

18. The method for optical analysis of claim 2 further comprising the step of detecting rotational transitions.

19. The method for optical analysis of claim 1 wherein the step of scanning a surface for a chemical further comprises scanning said surface for a trace chemical.

20. The method for optical analysis of claim 1 wherein the step of scanning a surface for a chemical further comprises scanning said surface for a bulk chemical.
